(19) United States
(12) Patent Application Publication
(10) Pub. No.: US 2009/0177076 A1
(43) Pub. Date: Jul. 9, 2009

(54) MRI OF A CONTINUOUSLY MOVING OBJECT INVOLVING MOTION COMPENSATION

(75) Inventors: Bernd Aldefeld, Hamburg (DE); Peter Boernert, Hamburg (DE); Jochen Keupp, Hamburg (DE); Johan Samuel Van Den Brink, Eindhoven (NL)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/296,936

(22) PCT Filed: Apr. 5, 2007
(86) PCT No.: PCT/IB07/51227

§ 371 Date: Oct. 13, 2008

(30) Foreign Application Priority Data
Apr. 13, 2006 (EP) 06112623.1

(51) Int. Cl. A61B 5/05

(52) U.S. Cl. 600/410

(57) ABSTRACT

A magnetic resonance examination system has an object carrier (14) to move an object to be examined relative to the field of view. A monitoring system (33) monitors examination circumstances under which magnetic resonance signals are acquired from the object within the field of view. In particular the monitoring system monitors the degree of physiological motion in the patient to be examined. A velocity control system (32) to control the velocity of the movement of the object relative to the field of view and to control the velocity on the basis of the monitored examination circumstances, i.e. the degree of physiological motion.

The invention pertains to a magnetic resonance examination system which has the function of examining a continuously moving object. Magnetic resonance examination involves the gathering of data on the basis of magnetic resonance techniques from the object to be examined and includes magnetic resonance imaging, but also includes spatially resolved magnetic resonance spectroscopy. In these applications the object is notably a human or animal patient to be examined. These magnetic resonance examinations for example make available information on the morphology of anatomical tissue or on the physiological functions of the body of the patient to be examined. There is a general need of magnetic resonance imaging of an object that is larger than the available field of view of the magnetic resonance imaging system. Further, imaging of a continuously moving object is considered to be more advantageous notably with respect to speed of acquisition and patient comfort than moving the object in large steps to a number of stations, acquiring data while the object is at rest and concatenate the images obtained at the individual stations to form the image of the object.

A magnetic resonance examination system for imaging of a continuously moving object is known from the international application WO2005/111649.

The cited document discloses a magnetic resonance imaging system in which image data from the object are acquired while the object is moving at a variable speed relative to the magnetic resonance imaging system. From the acquired image data an image of the object is reconstructed. The known magnetic resonance imaging system acquires image data relating to a central portion of k-space when the object is displaced through the field of view at low velocity while image data relating to a peripheral region in k-space are acquired when the object moves at high velocity. In this way, efficient data acquisition is achieved because the known magnetic resonance imaging system continues to acquire image data during periods of fast movement of the object. On the other hand, the known magnetic resonance imaging system produces magnetic resonance images having a low degree of image artefacts because image data from the central region of k-space, which are more susceptible from motion artefacts are acquired during periods of at best slow motion of the object.

An object of the invention is to provide a magnetic resonance examination system, which has a high efficiency of acquiring magnetic resonance data from an object that moves relative to the magnetic resonance examination system and in which perturbations in the acquired magnetic resonance data are further avoided.

This object is achieved by the magnetic resonance examination system of the invention having
a field of view and comprising
an object carrier to move an object to be examined relative to the field of view
a monitoring system to monitor examination circumstances under which magnetic resonance signals are acquired from the object within the field of view
a velocity control system to control the velocity of the movement of the object relative to the field of view and to control the velocity on the basis of the monitored examination circumstances.

The magnetic resonance examination system of the invention has a main magnet system to apply a static magnetic field through an examination region. Further the magnetic resonance examination system has an RF excitation system to transmit an RF excitation field into the examination region to excite (nuclear or electron) spins in the object to be examined. The excited spins cause emission of magnetic resonance signals. An object carrier is provided to support the object to be examined and pass the object through the examination region. Notably, the object carrier moves the object through the field of view during acquisition of magnetic resonance signals. Further a gradient system is provided to apply magnetic gradient fields, which cause spatial encoding of the magnetic resonance signals. Notably read gradients and/or phase encoding gradients are employed for spatial encoding. The acquisition of magnetic resonance data is performed by sampling magnetic resonance date in k-space in that magnetic resonance signals are acquired where the wave vector (k-vector) of the magnetic resonance signals is varied due to the application of temporary read gradients and/or phase encoding gradients (i.e. by application of corresponding encoding gradient waveforms or pulses). The magnetic resonance signals are acquired from the field of view. The highest magnitude of the k-vector determines the smallest wavelength of the acquired magnetic resonance signals and thus the resolution of e.g. the reconstructed magnetic resonance image. The largest wavelength corresponds to the smallest sampling step in k-space and is set in accordance with the field of view in the read direction and the phase encoding directions at issue so as to avoid fold-over artefacts. Hence the way k-space is sampled is determined by the application of the encoding gradient fields which are set in accordance with the field of view to achieve an acceptably low level of folding artefacts. The field of view is located within the examination region. Usually the examination region has a very high degree of spatial uniformity and temporal stability of the static magnetic field and the RF transmit field. Further, in the examination region the gradient fields have a high degree of linearity.

An insight of the present invention is that perturbation of the acquired magnetic resonance data is often related to the examination circumstances under which the magnetic resonance data are acquired. Perturbations of the magnetic resonance image can be caused by physiological motion or other signal changing phenomena. Notably, these examination circumstances involve physiological motion which is motion that is localised to a specific region of the body of the patient to be examined, or internal movements within the object this motion of a part of the object which is to be distinguished from the displacement of the object through the examination region (often indicated as the ‘table motion’). Particular examples of motion of a part of the object are displacement of a limb (arm or leg) of a patient to be examined or movement of the patient's head. Internal movements are for example respiratory motion caused by breathing of the patient to be examined or cardiac motion caused by the patient's heartbeat. The monitoring system monitors the examination circumstances, for example the amount of local motion. The velocity of the displacement of the object through the field of view is adjusted on the basis of the monitored examination circumstances. Thus, the velocity of the displacement is optimised according to the prevailing examination circumstances. In particular, the magnitude of the velocity is adjusted in dependence of the degree of motion that is monitored. For example the degree of motion involves the speed by which an anatomical structure such as an organ is moved and/or the distance over which the organ has moved. At a high degree of motion the velocity is lowered so that there is created ample opportunity to discard magnetic resonance signals that are likely to be corrupted or actually are corrupted e.g. due to motion artefacts and re-acquire better quality magnetic resonance signals and accordingly sample a sufficient region of k-space, often indicated as the ‘full k-space’ to acquire data from which the magnetic resonance image with a pre-set spatial resolution can be reconstructed. It is noted that when parallel imaging techniques are employed the sampling density of k-space may be lower than what is required to achieve, by (inverse) Fourier transformation, the pre-set spatial resolution. In practice a lower displacement velocity is employed when the degree of motion exceeds a threshold value. This threshold may be flexible and can be set by the operator or on the basis of previous experience. In addition, the threshold may be adjusted to different values for respective parts of the anatomy or to a fraction of the k-space to be sampled. This adjustment of the threshold can be made on the basis of the expected amount of motion in the part of the anatomy at issue. Accordingly the invention avoids/reduces artefacts in the magnetic resonance image due to motion in or of the patient, separate from the movement that is due to the displacement of the patient to be examined through the field of view.

For example the monitoring system makes use of so-called navigator signals to monitor the amount of motion, which can be determined by external sensors like the ECG, dilatation measuring belts, ultra sound sensors or MRI means. A special navigator signal can be generated by performing a local, e.g. pencil beam shaped RF excitation and receiving non-phase encoded magnetic resonance signals due to this local excitation. Such a navigator may be employed to sense respiratory motion at the patient's diaphragm. In effect such a respiratory navigator monitors the transition between liver and lung tissue, which accurately represents the movement of the patient's diaphragm due to respiratory (and cardiac) motion. In accordance with a further aspect of the invention the pencil beam excitation(s) of the navigator process are moved along with the displacement of the patient to be examined through the field of view, so that the position of the pencil beam excitation(s) is maintained stationary with respect to the patient's anatomy or with the table position.

These and other aspects of the invention will be further elaborated with reference to the embodiments defined in the dependent Claims.

According to a particular aspect of the invention the velocity of the object relative to the field of view is adjusted taking into account that the acceleration or deceleration is finite when the velocity of the object relative to the field of view is changed. Because the acceleration and deceleration are finite, the excited region is displaced in the time that lapses when the velocity is changed between a higher and a lower value. In particular the lower velocity is set still lower than what is needed to take account of only for a longer required signal acquisition time (i.e. when examining circumstance deteriorate) but also to account of the displacement during acceleration and/or deceleration. Taking into account the displacement during acceleration and/or deceleration is practically carried out on the basis of the acceleration and/or deceleration which are simply computed from the kinematics that is determined from data that are easily available from the motor drive which drives the object carrier through the field of view.

Further, the change of the velocity of the displacement of the object, that is deceleration and acceleration, may be effected when the body of the patient to be examined during its displacement reaches or leaves, respectively, an anatomical area where a high degree of motion is expected. For example, the degree of motion is expected to exceed the threshold value when the thorax or the abdomen reaches the field of view. There are various ways to determine that an anatomical area where a high degree of motion is likely to occur reaches of leaves the field of view. For example a ‘scout scan’ that is acquired a priori, which provides coarse outlines of various parts of the anatomy and which requires only a low spatial resolution may be employed. As an alternative, one or more pencil beam acquisitions may be employed to detect edges. These detected edges are correlated with edge information from an anatomical model. The degree of motion represents for example the speed of the local motion and/or the distance over which such motion occurs.

According to a further aspect of the invention the magnitude of the velocity of the object is set in dependence of the required signal acquisition time and the size of the excited region, notably the width of the excited region in the direction of the movement of the object relative to the field of view. The relative velocity between the object and the field of view is set such that during the required signal acquisition time the object travels at most the width of the excited region in the direction of movement. The required signal acquisition time is the time needed to acquire magnetic resonance signals to cover the region of k-space that corresponds with the pre-required spatial resolution of the magnetic resonance image and the field of view, i.e. the full k-space at issue. An efficient acquisition of magnetic resonance signals from the object is achieved when the object travels exactly the width of the excited region during the required signal acquisition time. In this implementation of the invention magnetic resonance signals acquired for successive scans of the moving excited region seamlessly match as the object moves relative to the field of view. On the one hand, when the velocity is set somewhat lower, then remaining time is available to acquire additional magnetic resonance signals, the ensuing redundancy may be employed to improve image quality of the reconstructed magnetic resonance image. For example residual artefacts may be corrected for or noise can be reduced by some averaging. On the other hand, when the velocity is set somewhat higher, then some magnetic resonance signals that are missed because the excited region has moved too far through the field of view, the missing data can be restored by computation from the data that are actually acquired e.g. by interpolation or extrapolation.

In a further aspect of the invention the RF excitation system moves the excited region synchronously with the movement of the object. Notably, the excited region is moved by adjusting the carrier frequency of the RF excitation field so that the excited region moves in which the excited spins e.g. nuclear spins or electron spins) are in resonance with the RF excitation field, in conjunction with the magnetic gradient field. According to this aspect of the invention several parts of trajectories in k-space, e.g. lines in k-space or parts of spiral arms, in k-space are scanned to acquire magnetic resonance signals from essentially the same physical positions in the object as it moves through the field of view. Often the excited region has the shape of a slab that moves through the field of view and k-space is scanned once for the whole slab when the slab moves from its begin position to its end position. In this way it is possible to image objects that are larger than the size of the field of view, while the object moves continuously through the field of view.

According to another aspect of the invention the examination circumstances are monitored. Particular examples of the examination circumstances are the level of motion of the object and/or movement occurring in the object. The examination circumstances describe whether good quality magnetic resonance signals can be expected to be acquired. Notably, as there is a higher level of motion, magnetic resonance signals are likely to be corrupted in that they contain more motion artefacts. Preferably, the velocity of the object relative to the field of view is lowered as examination circumstances are worse, e.g. higher degree of motion occurs. Acquisition of magnetic resonance signals may be interrupted and/or magnetic resonance signals that are severely corrupted are rejected. In another implementation the MR acquisition pulse sequence continues, but when examination circumstances deteriorate, dummy cycles may be applied in which there is no signal read out, or signals read out are not accepted. Continuation of the MR acquisition pulse sequences maintains the magnetisation state of the object, so that notably in steady state sequences, such as balanced-FFE the steady state magnetisation is not or hardly affected. Because the velocity is relatively low, it is yet achieved that magnetic resonance signals acquired for successive scans of the moving excited region seamlessly match as the object moves relative to the field of view.

In general, the invention relates to magnetic resonance imaging methods in which k-space data are acquired directly while the patient table moves. Physiological motion of the patient, such as breathing, is detected e.g. by a patient-motion sensor, and the MR sequence is gated so that k-space data are accepted only for certain motion states. The table velocity is changed during the scan so that conformity with the gated MR sequence is always maintained. The exact position of the patient table is measured by a table-position sensor. Its output information is used in the reconstruction unit for reproducing the data origin so as to achieve exact matching of the anatomy with the sampled data. Excess scan time requirement due to gating is confined to regions where physiological motion is significant, whereas other regions (head, lower body) can be scanned at normal table velocity. In particular, respiratory and/or cardiac gating may be employed to accept magnetic resonance signals only when respiratory or cardiac motion, respectively is within a preset gating window. It is noted that the gating window may be adapted to the region of k-space being scanned in that for magnetic resonance signals from a centre region of k-space the gating window is set to a narrow range and for magnetic resonance signals from a peripheral region of k-space the gating window is set to a wider range. Further, the acceptance window may be set in dependence of the position of the patient's body in the examination region, i.e. the part of the anatomy that is actually scanned. The invention achieves as its main advantages that whole-body imaging, such as cancer screening, is made possible without loss of image quality (e.g. blurring) in the abdominal region.

The invention also pertains to a magnetic resonance imaging method as defined in claim 9. The method of the invention achieves efficient data acquisition in combination with a low (motion) artefact level for magnetic resonance imaging of a continuously moving object.

It is noted that the method of the invention solves the technical problem of reducing the level of image artefacts notably due to internal motion in or of the patient to be examined. The resulting magnetic resonance image(s) are useful for the medical practitioner to assess the physical condition of the patient to be examined. That is, the resulting magnetic resonance image(s) form a starting point for the medical practitioner to engage in the intellectual medical diagnostic deduction phase that is to take place subsequent to the methods steps of the claimed method. Moreover, the medical diagnostic deduction phase does not require physical interaction with the patient to be examined.

The invention also pertains to a computer programme as defined in claim 10. The computer programme of the invention can be provided on a data carrier such as a CD-rom disk, or the computer programme of the invention can be downloaded from a data network such as the worldwide web. When installed in the computer included in a magnetic resonance imaging system the magnetic resonance imaging system is enabled to operate according to the invention and achieves efficient data acquisition in combination with a low (motion) artefact level for magnetic resonance imaging of a continuously moving object

These and other aspects of the invention will be elucidated with reference to the embodiments described hereinafter and with reference to the accompanying drawing wherein

FIG. 1 presents an overview of the method of the invention as a schematic drawing;

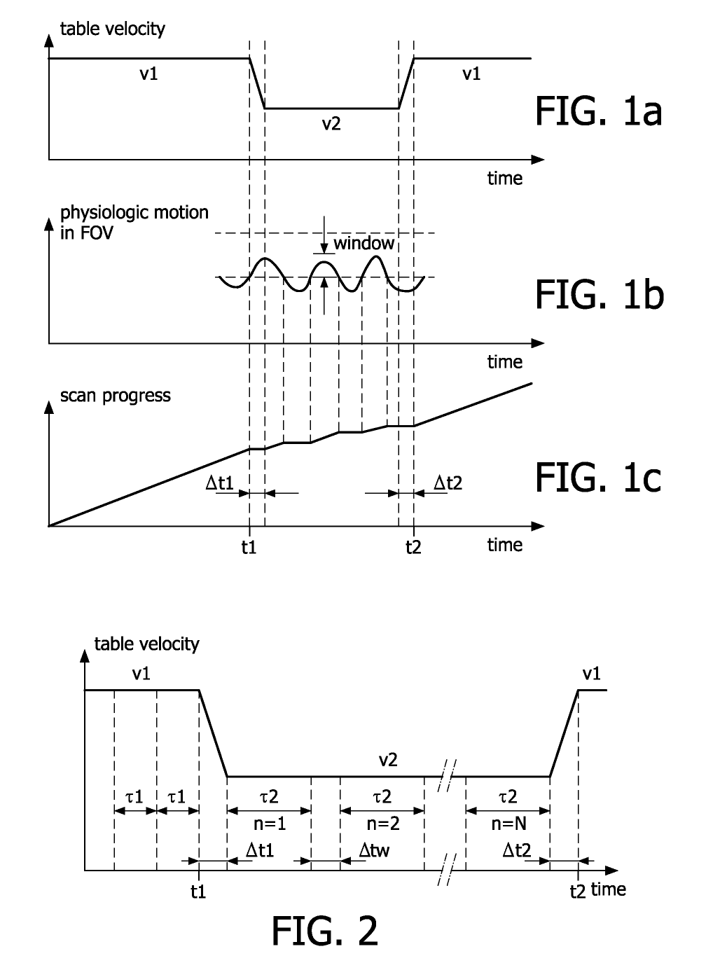

FIG. 2 presents the timing of the slab motion with the MR sequence which is important for image quality;

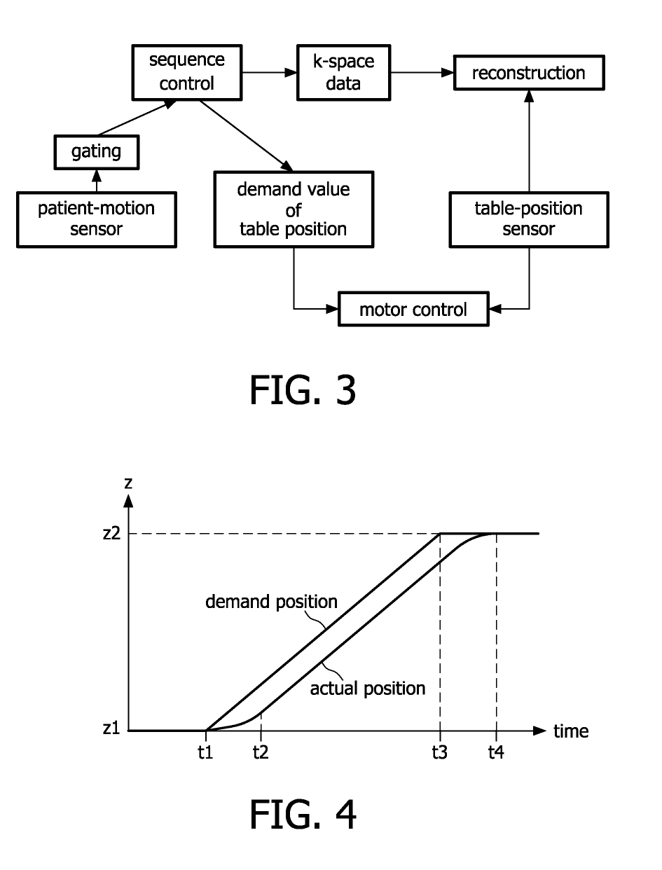

FIG. 3 illustrates an implementation of the invention;

FIG. 4 illustrates an example for a smooth table movement and

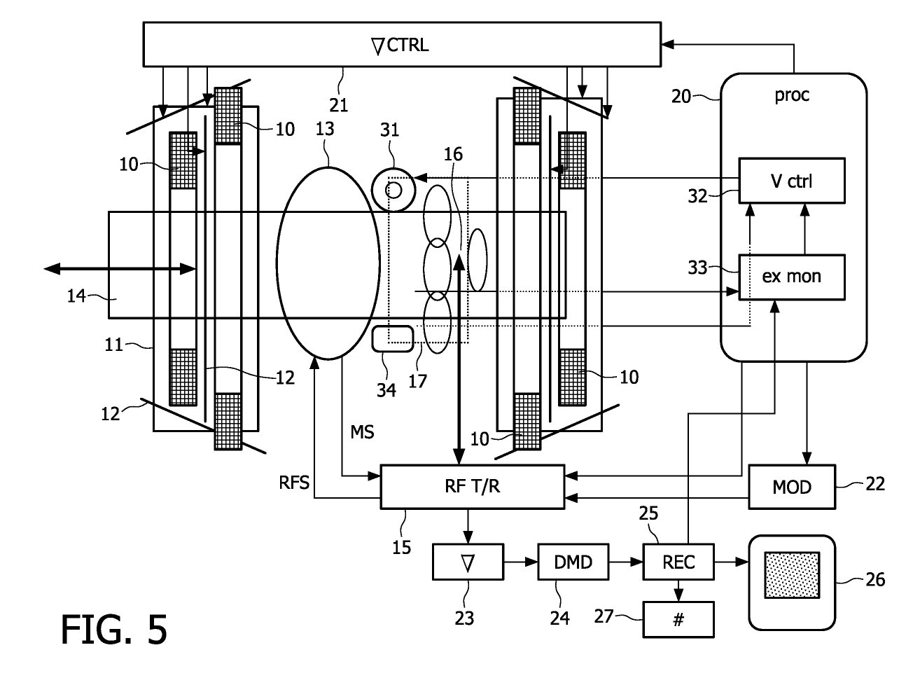

FIG. 5 shows diagrammatically a magnetic resonance imaging system in which the invention is used.

FIG. 5 shows diagrammatically a magnetic resonance imaging system in which the invention is used. The magnetic resonance imaging system includes a set of main coils 10 whereby the steady, uniform magnetic field is generated. The main coils are constructed, for example in such a manner that they enclose a tunnel-shaped examination space. The patient to be examined is placed on a patient carrier which is slid into this tunnel-shaped examination space. The magnetic resonance imaging system also includes a number of gradient coils 11, 12 whereby magnetic fields exhibiting spatial variations, notably in the form of temporary gradients in individual directions, are generated so as to be superposed on the uniform magnetic field. The gradient coils 11, 12 are connected to a gradient control 21 which includes one or more gradient amplifier and a controllable power supply unit. The gradient coils 11, 12 are energised by application of an electric current by means of the power supply unit 21; to this end the power supply unit is fitted with electronic gradient amplification circuit that applies the electric current to the gradient coils so as to generate gradient pulses (also termed ‘gradient waveforms’) of appropriate temporal shape The strength, direction and duration of the gradients are controlled by control of the power supply unit. The magnetic resonance imaging system also includes transmission and receiving coils 13, 16 for generating the RF excitation pulses and for picking up the magnetic resonance signals, respectively. The transmission coil 13 is preferably constructed as a body coil 13 whereby (a part of) the object to be examined can be enclosed. The body coil is usually arranged in the magnetic resonance imaging system in such a manner that the patient 30 to be examined is enclosed by the body coil 13 when he or she is arranged in the magnetic resonance imaging system. The body coil 13 acts as a transmission antenna for the transmission of the RF excitation pulses and RF refocusing pulses. Preferably, the body coil 13 involves a spatially uniform intensity distribution of the transmitted RF pulses (RFS). The same coil or antenna is usually used alternately as the transmission coil and the receiving coil. Furthermore, the transmission and receiving coil is usually shaped as a coil, but other geometries where the transmission and receiving coil acts as a transmission and receiving antenna for RF electromagnetic signals are also feasible. The transmission and receiving coil 13 is connected to an electronic transmission and receiving circuit 15.

It is to be noted that it is alternatively possible to use separate receiving and/or transmission coils 16. For example, surface coils 16 can be used as receiving and/or transmission coils. Such surface coils have a high sensitivity in a comparatively small volume. The receiving coils, such as the surface coils, are connected to a demodulator 24 and the received magnetic resonance signals (MS) are demodulated by means of the demodulator 24. The demodulated magnetic resonance signals (DMS) are applied to a reconstruction unit. The receiving coil is connected to a preamplifier 23. The preamplifier 23 amplifies the RF resonance signal (MS) received by the receiving coil 16 and the amplified RF resonance signal is applied to a demodulator 24. The demodulator 24 demodulates the amplified RF resonance signal. The demodulated resonance signal contains the actual information concerning the local spin densities in the part of the object to be imaged. Furthermore, the transmission and receiving circuit 15 is connected to a modulator 22. The modulator 22 and the transmission and receiving circuit 15 activate the transmission coil 13 so as to transmit the RF excitation and refocusing pulses. In particular the surface receive coils 16 are coupled to the transmission and receive circuit by way of a wireless link. Magnetic resonance signal data received by the surface coils 16 are transmitted to the transmission and receiving circuit 15 and control signals (e.g. to tune and detune the surface coils) are sent to the surface coils over the wireless link.

The reconstruction unit derives one or more image signals from the demodulated magnetic resonance signals (DMS), which image signals represent the image information of the imaged part of the object to be examined. The reconstruction unit 25 in practice is constructed preferably as a digital image-processing unit 25 which is programmed so as to derive from the demodulated magnetic resonance signals the image signals which represent the image information of the part of the object to be imaged. The signal on the output of the reconstruction monitor 26, so that the monitor can display the magnetic resonance image. It is alternatively possible to store the signal from the reconstruction unit 25 in a buffer unit 27 while awaiting further processing.

The magnetic resonance imaging system according to the invention is also provided with a control unit 20, for example in the form of a computer which includes a (micro)processor. The control unit 20 controls the execution of the RF excitations and the application of the temporary gradient fields. To this end, the computer program according to the invention is loaded, for example, into the control unit 20 and the reconstruction unit 25.

In accordance with the invention, the magnetic resonance examination system is provided with a motor drive 31, which drives the object carrier 14 along the directions indicated by the double arrow through the field of view 17. The motor drive is controlled by the velocity control system 32 that is included in the processor 20. The velocity control system regulates the motor drive 31 to drive the object carrier at the appropriate velocity. Further a monitoring system is provided which monitors the examining circumstances. For example the monitoring system may detect respiratory motion of the patient to be examined from a respiratory belt strapped around the patient's chest. Alternatively motion may be detected in general on the basis of the acquired magnetic resonance signals or reconstructed image information; e.g. on the basis of navigator technique. In an other implementation cardiac motion may be derived using an ECG or on the basis of a navigator technique or tagging. A table position sensor 34 is provided to assess the actual position of the object carrier 14. The actual table position is provided to the velocity control system 32 to set the velocity at which the motor drive 31 drives the object carrier in dependence of the portion of the anatomy being in the field of view. Notably the velocity control system 32 and the monitoring system 33 are in practice implemented in software. In continuously moving table imaging, the motion of the patient table can be compensated by adequate computational method, as described e.g. in reference [1]. However, motion of the patient's body such as breathing (physiological motion) poses a problem because this results in degraded images. In static (without table motion) MRI, images can sometimes be acquired during a breath-hold of the patient. But this measure can normally not be applied in moving table applications because the acquisition time is typically much too long for a breath-hold. Thus, MR image data must be acquired while the patient breathes. In MR examinations in which a large part of the anatomy is covered, e.g. head-to-toe cancer screening, the breathing problem exists only during a certain fraction of the total scan, i.e. when the abdominal and chest regions are in the useful FOV of the scanner. The problem does not exist, or is negligible, at times when body parts other than the abdominal and chest regions are within the useful FOV of the scanner.

From static MRI, methods for the reduction of the effects of physiological motion are already known, such as methods based on motion sensors and gating of the MR sequence. With this method, data are accepted only when the output signal of the motion sensor falls into a predefined window of acceptance. When such techniques are to be applied in the context of continuously moving table imaging, it is important to restrict the gating to only that part of the data acquisition where it is necessary, else the total scan time would be unnecessarily prolonged. Further, since the time to scan k-space is increased by the use of gating, it is important to allow two or more different table velocities during a single MR scan, and to adjust the data acquisition process accordingly.

It is thus the aim of the present invention to allow continuously moving table imaging of an extended anatomy, compensating the patient's physiological motion using variable table velocity and signal gating preferably during only part of the scan.

An overview of the method of the invention is presented with reference to schematic drawing FIG. 1, which illustrates only the basic method. It is assumed here that the scan starts and ends without gating, and that gating is confined to one time interval. The table velocity (FIG. 1a) is v₁=constant up to time t1, decreases during the interval Δt1 to the value v₂, and increases again to the value v₁ during the interval Δt₂. The time needed for acceleration or deceleration is easily computed from the capacity of the table motor. The higher velocity, v₁, is chosen during times when physiological motion of the patient does not occur inside the field of view (FOV), e.g. when the head or lower-extremity regions are in the FOV. The slower velocity, v₂, however, is chosen when physiological motion of the patient becomes significant within the FOV, e.g. when the abdominal region is in the FOV.

FIG. 1b illustrates the output signal of the sensor that monitors the physiological motion of the patient during the time when such motion may disturb the data acquisition (sensitive time interval). The deceleration of the velocity coincides with the start of the sensitive time interval (time t₁), and the acceleration starts near the end of the sensitive time interval (time t₂). The physiologic motion is detected by one or more sensors, e.g. respiratory belts, or navigator pulses interleaved into the MR sequence. A motion sensor may be active over a wider range than indicated in FIG. 1. However, its output affects the data acquisition process only when physiological motion occurs within the FOV.

FIG. 1c illustrates the progress of the data acquisition process. As long as the table is moved at velocity v₁, data are continuously acquired since physiological motion is not relevant here. While the velocity is v₂, data are acquired only when the amplitude of the physiological motion falls into the predefined window of acceptance, else no data are acquired. The data acquisition may be interrupted during periods of acceleration or deceleration, as shown in the example FIG. 1c, or it may continue during these intervals. After the velocity is equal to v₁ again, normal continuous data acquisition is resumed.

In FIG. 1, velocity v₂ was assumed constant, for simplicity. This is not, however, a necessary condition, as will become clear from the following two embodiments of the basic idea. In embodiment I, constant velocity v₂ is assumed, whereas the velocity v₂ may vary in embodiment II.

### Embodiment I

An MR sequence is used in which a slab of volume with length L along the direction of motion is excited by the RF pulse. The RF excitation is varied such that the slab is moved in the FOV from a start to an end position synchronously with the motion of the patient table. While the slab moves from the start to the end position, k-space is scanned once. After each such scan of k-space, an anatomical region of length L can be reconstructed in known manner. The cycle is then repeated several times until the desired length of the anatomy has been scanned. The timing of the slab motion with the MR sequence is important for image quality and will be described in the following, with reference to FIG. 2.

From the start of the sequence up to time t₁, the data acquisition proceeds as is common practice in continuously moving table imaging, i.e. several scans of k-space are performed in succession at constant velocity v₁. The sequence parameters are chosen such that the fastest data acquisition (no averaging) is assured:

τ₁×v₁=L (1)

where τ₁ denotes the time to scan k-space exactly once. At time t₁, physiological motion becomes important. The table velocity is decreased to the value v₂ during time Δt₁, while no data are acquired. One or more scans of k-space, n=1 . . . N, are then performed at velocity v₂, each requiring time t₂. At time t₂−Δt₂, the table velocity is increased to v₁ again, and the scan proceeds as before time t₁. During the time intervals Δt_{w} between the individual scans n=1 . . . N−1, no data are acquired. These intervals serve to guarantee the matching condition between the MR sequence and the motion of the patient table. It is assumed that only a fraction f (gating efficiency) of k-space data are accepted during τ₂ as given by the window of acceptance (see FIG. 1). Thus

τ₂=τ₁/f (2)

Velocity v₂ and the time intervals Δt₁, Δt₂ and Δt_{w} are unknown and must be determined for proper control of the sequence.
During deceleration from v₁ to v₂, the table moves the distance Δz. During the following scan of k-space (duration τ2), the table moves the distance v₂×τ₂. To ensure that a seamless image is acquired, the table displacement including the transition time should be equal to L, i.e. the equation to be satisfied is

Δz+v₂×τ₂=L (3)

The length Δz over which the table is decelerated can be computed from the table-motor data. Assuming, for example, constant deceleration

$$a = \frac{d v}{d t} = const \qquad ( 4 )$$

then

Δz=v₁×Δt₁−½×a×Δt₁² (5)

and further from Eq. (4)

$$\begin{matrix}Δ {t}_{1} = \frac{{v}_{1} - {v}_{2}}{a} \\ so that & ( 6 ) \\ Δ z = \frac{{v}_{1} ( {v}_{1} - {v}_{2} )}{a} - \frac{{( {v}_{1} - {v}_{2} )}^{2}}{2 a} & ( 7 )\end{matrix}$$

Using this expression and Eq. (2) in Eq. (3), we obtain

$$\frac{{v}_{1} ( {v}_{1} - {v}_{2} )}{a} - \frac{{( {v}_{1} - {v}_{2} )}^{2}}{2 a} + \frac{{v}_{2} {τ}_{1}}{f} = L \qquad ( 8 )$$

Solving this equation, velocity v₂ is obtained as

$$\begin{matrix}{v}_{2} = A - \sqrt{B + {A}^{2}} \\ where & ( 9 ) \\ A = \frac{a {τ}_{1}}{f} ; & ( 10 ) \\ B = {v}_{1}^{2} - 2 aL & ( 11 )\end{matrix}$$

After the velocity v₂ has been computed using Eqs. (9,10,11), the deceleration time Δτ₁ is obtained from Eq. (6). The acceleration time Δτ₂, at the end of which velocity v₁ is reached again, can either be computed in analogy with Eq. (6), using a different motor acceleration if desired, or it can be set equal to Δτ₁. The waiting time Δt_{w} between the k-space scans n=1 . . . N−1 (FIG. 2) can be computed from the condition that the slab should have moved the distance L after each complete scan of k-space including the waiting time, i.e.

v₂×(τ₂+Δτ_{w})=L (12)

from which

Δτ_{w}=L/v₂−τ₂ (13)

is obtained. Note: In the above equations, it was assumed that n>2. Obvious modifications are obtained for n=1. The time intervals Δt₁, Δt₂ and Δt_{w} are normally very small compared with τ1 and τ2.

The assumption expressed in Eq. (2) above is that the gating efficiency f is known. In practice, the gating efficiency depends on several factors, e.g. the breathing rhythm of the patient. It may thus happen that its value is not estimated properly. If the assumed value off is too low, then k-space is covered in a time period shorter than τ₂. The remaining time can then be used, for example, to acquire additional k-space data, or to measure and update other data such as the resonance frequency, or to simply apply dummy cycles to maintain the steady state. If the assumed value off is too high, then some k-space data are missed during the interval τ₂. In this case, the missing data can be computed by interpolation or extrapolation based on the data acquired. To avoid this situation one can acquire all k-space data in the first run ignoring the gating information, but bookkeeping the neglected gating decisions to subsequently perform re-acquisition of unacceptable data. In this way in any case data are available for reconstruction, even if they are corrupted, and extrapolation is not necessary. During the reacquisition process motion adapted gating [7] could be applied to perform for instance a k-space depended gating for improving image quality.

In general the simple accept/reject gating procedure mainly used so far, can be replaced by more advanced gating concepts in combination with continuously moving table imaging.

### Embodiment II

The main feature is that the motion of the patient table during the sensitive time interval is directly controlled by the progress of the data acquisition. Starting point is the requirement that for each line of k-space acquired, the patient table must move the distance

$$Δ L = \frac{L}{P} \qquad ( 14 )$$

where P denotes the total number of lines in k-space. This ensures that the patient table has traveled the distance L for each full coverage of k-space, and a seamless image is acquired. In order to avoid jerky table motion, the motion of the patient table may be delayed relative to its required position. The method will explained in more detail with reference to FIG. 3.

Lines of k-space are acquired as determined by the patient-motion sensor and the adjustment of the gating window. Typically, lines of k-space are acquired in blocks of several lines, but this is not a necessary condition. For each acquired line of k-space, the demand value of the table position is increased by ΔL according to Eq. (14). A table-position sensor measures the actual position of the patient table, which may be different from the demand value. The demand and actual values of the table position are send to a motor control unit, which computes a smooth path that closely follows the demand path and steers the motor accordingly. The actual table position for each acquired line of k-space is also send from the table-position sensor to the reconstruction unit, so that each line of k-space can be associated with the correct portion of the examined anatomy.

An example for a smooth table movement is illustrated in FIG. 4. It is assumed here that a block of m lines of k-space is acquired in the time interval from t₁ to t₃, starting at the table position z₁. At time t₃, the demand table position, relative to z₁, is given by Eq. (14) for m lines of k-space:

$${z}_{2} - {z}_{1} = \frac{mL}{P} \qquad ( 15 )$$

If the table could move at velocity

$${v}_{0} = \frac{{z}_{2} - {z}_{1}}{{t}_{3} - {t}_{1}} \qquad ( 16 )$$

between t₁ and t₃, then the demand position would be satisfied at any point in time, but this would require infinite acceleration. Thus, an acceleration and a deceleration interval are incorporated, and smooth table motion is obtained by control of the motor as follows:

v=a×t from t₁ to t₂

v=v₀ from t₂ to t₃ (17)

v=v₀−a×(t−t₃) from t₃ to t₄

with e.g. constant acceleration a. The actual table position coincides with the demand table position only at the end of the acquisition of m lines of k-space. Nevertheless, exact reconstruction is guaranteed because the actual table position is measured and that information is used by the reconstruction algorithm. Depending on the average velocity of table motion, the difference between the demand and actual table positions may be very small. In this case, a table-position sensor may not be necessary.

Each of the above embodiments can be varied to satisfy the specific requirements of the MR sequence considered. In particular, several sections with different velocities can be used in embodiment I. Also, acceleration and deceleration times need not be equal. Further, the above equations can be modified such that critical time intervals are always multiples of the repetition time.

One possible realization of a patient-motion sensor employs local navigator beams and receiving non-phase encoded magnetic resonance signals due to this local excitation. Here, a signal from e.g. the diaphragm area is repeatedly detected and compared with a reference signal. This method, however, would fail in moving-table imaging unless adequate modifications are applied. One possible modification for this purpose is to synchronize the position of the navigator beam with the motion of the table, in analogy with the slab-tracking method. That is, the position of the navigator beam is advanced in the direction of table motion such that it is fixed to the anatomy for some period of time, after which it is reset to a start position. Also a navigator that extends along the direction of displacement of the patient through the field of view my be employed. Such a navigator that extends along the direction of displacement may be stationary with respect to the examination region. This navigator is useful to account for so-called ‘relaxing’ of the patient during the examination.

The above method has been described with particular reference to the Cartesian type of scanning. This, however, is not a necessary condition. With obvious modifications, the method can also be applied to radial and spiral scanning schemes.

### REFERENCES

[1] D. G. Kruger and S. J. Riederer, Method for acquiring MRI data from a large field of view using continuous table motion, US2002/0173715A1, Nov. 21, 2002.
[2] J. V. Hajnal, Magnetic resonance imaging apparatus, EP1024371A2, 1999.
[3] J. H. Brittain, Moving table MRI with frequency encoding in the z-direction, US2002/0140423A1, Oct. 3, 2002.
[4] C. A. Mistretta, Magnetic resonance angiography using floating table projection imaging, U.S. Pat. No. 6,671,536B2, Dec. 30, 2003.
[5] D. G. Kruger, et al, A dual-velocity acquisition method for continuously-moving-table contrast-enhanced MRA, Proc ISMRM 2004, Tokyo, p. 233.
[6] J. Ma, Whole body MRI at 3 Tesla using a moving tabletop and a fast spin-echo Dixon technique, Proc ISMRM 2005, Miami, p. 1965.
[7] R. Sinkus, P. Bornert P. Motion pattern adapted real-time respiratory gating. Magn Reson Med. 1999; 41:148-55.

### LIST OF SYMBOLS

| | |
|---|---|
| a | acceleration of table |
| f | gating factor |
| L | field of view from which data are sampled |
| P | number of lines in k-space |
| t | time |
| v | velocity of table |
| t | time required for one scan of k-space |
| z | coordinate along table motion |

1. A magnetic resonance examination system having
a field of view and
comprising
an object carrier to move an object to be examined relative to the field of view
a monitoring system to monitor examination circumstances under which magnetic resonance signals are acquired from the object within the field of view
a velocity control system to control the velocity of the movement of the object relative to the field of view and to control the velocity on the basis of the monitored examination circumstances.

2. A magnetic resonance examination system as claimed in claim 1, wherein the monitoring system is arranged to
determine acceptance or rejection of acquired magnetic resonance signals on the basis of the monitored examination circumstances,
supply an acceptance efficiency to the velocity control system and
the velocity control system is arranged to take account of the acceptance efficiency for adjustment of the velocity of the movement of the object.

3. A magnetic resonance examination system as claimed in claim 1, comprising an RF excitation system arranged to carry-out an RF excitation in an excited region such that the excited region is moved relative to the field of view synchronously with the movement of the object.

4. A magnetic resonance examination system as claimed in claim 1, wherein the velocity control system is arranged to adjust the velocity of the movement of the object on the basis of the size of the excited region and the required signal acquisition time to scan k-space to acquire magnetic resonance signals from which the excited region of the object can be reconstructed.

5. A magnetic resonance examination system as claimed in claim 1, wherein the velocity control system is arranged to take account of a finite acceleration involved in changing the velocity of the movement of the object for adjustment of the velocity of the movement of the object.

6. A magnetic resonance examination system as claimed in claim 1, wherein the examination circumstances include a degree of motion in at least a part of the object.

7. A magnetic resonance examination system as claimed in claim 1, wherein the velocity control system is arranged to drive the object to move at a higher velocity at a low degree of motion and at a lower velocity at a high degree of motion.

8. A magnetic resonance examination system as claimed in claim 1 in which the monitoring system is arranged to apply one or more navigators are applied to monitor movement in the object to be examined and wherein the location of the navigator excitation(s) is maintained relative to the moving object.

9. A magnetic resonance examination method involving
a field of view and comprising
displacing an object to be examined relative to the field of view
monitoring examination circumstances under which magnetic resonance signals are acquired from the object within the field of view
a velocity control system to control the velocity of the movement of the object relative to the field of view and to control the velocity on the basis of the monitored examination circumstances.

10. A computer programme comprising instructions to
displace an object to be examined relative to the field of view of an magnetic resonance examination system
monitor examination circumstances under which magnetic resonance signals are acquired from the object within the field of view
control the velocity of the movement of the object relative to the field of view and to control the velocity on the basis of the monitored examination circumstances.
